(19) United States
(12) Patent Application Publication
(10) Pub. No.: US 2009/0082827 A1
(43) Pub. Date: Mar. 26, 2009

(54) HINGED ANCHORS FOR WIRELESS PACING ELECTRODES

(75) Inventors: Graig Kveen, Maple Grove, MN (US); Douglas Saholt, Mound, MN (US); Joseph Thielen, Buffalo, MN (US); Mark Jenson, Greenfield, MN (US); William Drasler, Minnetonka, MN (US)

Correspondence Address: FAEGRE & BENSON, LLP;ATTN: PATENT DOCKETING (32469), 2200 WELLS FARGO CENTER, 90 SOUTH SEVENTH STREET, MINNEAPOLIS, MN, 55402-3901, US

(73) Assignee: CARDIAC PACEMAKERS, INC., St. Paul, MN (US)

(21) Appl. No.: 11/861,911

(22) Filed: Sep. 26, 2007

(51) Int. Cl. A61N 1/362

(52) U.S. Cl. 607/36

(57) ABSTRACT

A hinged anchor for a medical device electrode is disclosed. In one embodiment, the hinged anchor has a hinged portion and an anchor portion. The hinged portion can have a first configuration forming a first angle and a second configuration forming a second angle. The second angle can be a sharper angle than the first angle, and the hinged portion can be predisposed to assume the second configuration. The hinged anchor can be disposed on a control module of a leadless microstimulator device.

### TECHNICAL FIELD

The present invention relates to implantable medical devices for stimulating body tissues and/or sensing physiological attributes. More specifically, the invention relates to hinge structures for pacing and sensing electrodes.

### BACKGROUND

Various physiological functions can be managed and/or monitored using implantable medical devices. Implantable medical devices can have electrodes, and the electrodes can provide stimulating and/or sensing functionality to assist with a patient's health care. For example, implantable medical devices have been used in association with cardiac rhythm management, which can include cardiac pacing, cardiac defibrillation, and/or cardiac therapy, among other procedures. In some cases, such implantable medical devices can be fixed onto or into tissues of a patient. Various designs for fixing implantable medical devices onto or into tissues are known in the art. There exists a need for alternative designs and methods for fixing implantable medical devices onto or into tissues.

### SUMMARY

One embodiment of the invention comprises a leadless microstimulator comprising an anchor configured to penetrate and engage tissue at an implantation site, the anchor defining a first longitudinal axis. A control module, which defines a second longitudinal axis, is configured to generate an electrical stimulus. A hinge is disposed between the anchor and the control module which has a first configuration and a second configuration, wherein in the first configuration the first and second longitudinal axes are more closely aligned than in the second configuration. The hinge is predisposed to assume the second configuration and the hinge is deflectable between the first and second configurations.

Another embodiment of the invention has an electrode for a leadless microstimulator comprising a hinged anchor. The hinged anchor has an anchor portion and a hinged portion wherein the hinged portion has a first configuration and a second configuration. The hinged portion forms a first angle in the first configuration and a second angle in the second configuration, the second angle being sharper than the first angle. The hinged portion is predisposed to assume the second configuration.

In yet another embodiment of the invention, a microstimulator comprises a control module, an anchor portion and a flexible hinge. The flexible hinge is disposed between the control module and the anchor portion; and the hinge is predisposed to form a nonlinear configuration.

While multiple embodiments are disclosed, still other embodiments of the present invention will become apparent to those skilled in the art from the following description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows an exemplary leadless implantable medical device implanted in a heart according to embodiments of the present invention;

FIG. 2 shows a perspective view of a leadless electrode implanted in a portion of a heart according to embodiments of the present invention;

FIG. 3 shows a perspective view of an unimplanted leadless electrode of FIG. 2;

FIG. 4 shows a perspective view of another leadless electrode according to embodiments of the present invention;

FIG. 5 shows a perspective view of a hinged anchor according to embodiments of the present invention;

FIG. 6 shows a perspective view of a hinged anchor of FIG. 5 incorporated into a microstimulator according to embodiments of the present invention;

FIG. 7 shows a perspective view of the microstimulator of FIG. 6 in a second configuration;

FIG. 8 shows a longitudinal cross-sectional view of a delivery device according to embodiments of the present invention;

FIG. 9 shows an axial cross-sectional view of a delivery device according to embodiments of the present invention;

FIGS. 10A and 10B show two configurations of a delivery device according to embodiments of the present invention;

FIG. 11A shows a microstimulator in a delivery device according to embodiments of the present invention;

FIG. 11B shows the microstimulator of FIG. 11A implanted in heart tissue;

FIG. 12A shows a microstimulator in a delivery device according to embodiments of the present invention;

FIG. 12B shows the microstimulator of FIG. 12A implanted in heart tissue;

FIG. 13A shows a microstimulator in a delivery device according to embodiments of the present invention; and

FIG. 13B shows the microstimulator of FIG. 13A implanted in heart tissue.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

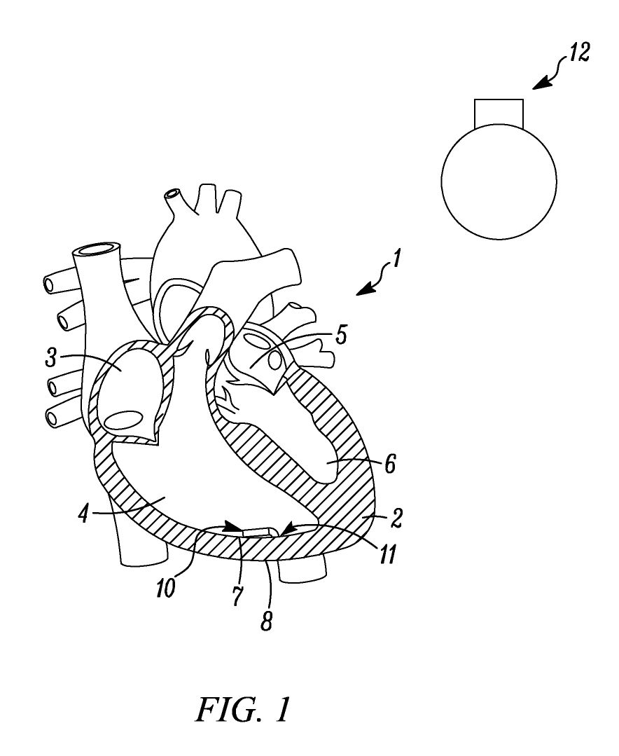

FIG. 1 is a combined cutaway view of a human heart 1 and a perspective view of an exemplary implantable medical device (IMD). The IMD includes a wireless microstimulator 10 and a remote module 12. The wireless microstimulator 10 can be coupled to the heart 1 at location 11. The heart 1 comprises a myocardium or cardiac muscle 2, a right atrium 3, and a right ventricle 4. The heart 1 further comprises a left atrium 5 and a left ventricle 6.

In the embodiment shown in FIG. 1, the microstimulator 10 is implanted in the right ventricle 4, and a portion of the microstimulator 10 is shown attached to the endocardium or inner layer 7 of the heart 1. The microstimulator 10 may both sense electrical signals of the heart 1 and/or deliver electrical pulses to the heart 1 in an attempt to correct an arrhythmia and restore sinus rhythm. In other embodiments, the microstimulator 10 can be implanted or placed within any chamber of the heart 1. For example, the microstimulator 10 can be implanted in the right atrium 3 or the left ventricle 6 or the left atrium 5. In addition, the microstimulator 10 can be implanted to or be placed on the epicardium 8, for example the epicardium 8 of the right ventricle 4, the right atrium 3, the left ventricle 6 or the left atrium 5. In such cases, the microstimulator 10 can be delivered through the circulatory system of the heart to the location of interest, or it can be implanted or placed on the epicardium 8 by gaining access to the pericardial space. In some embodiments, the microstimulator 10 may be implanted through the epicardium 8 or endocardium 7 and into the myocardium 2. In other embodiments, the IMD may include a plurality of microstimulators 10, each placed in, implanted in, or attached to a different chamber or a different part of the heart 1.

The implantable devices can be bipolar or unipolar. In a unipolar system, an electrode of the microstimulator 10 acts as one pole of an electrical system, and the second pole of the electrical system can be located remotely from the electrode. For example, the second pole of the electrical system can be located on the remote module 12, or it can be located in another portion of the patient's body or on the surface of the patient's body. Various other configurations for unipolar devices are known in the art.

In a bipolar system, the implantable device can have two electrodes disposed near the site of treatment. For example, a microstimulator 10 can have two electrodes disposed on the body of the microstimulator 10 (e.g., a tip electrode and a ring electrode disposed on the microstimulator 10 away from the tip electrode). The two electrodes can act as the two electrical poles of the microstimulator 10. Various other configurations for bipolar electrodes are known in the art.

When the implantable medical device is energized, an electrical potential can be created between the two electrical poles of the device. This potential can create an electrical field and, in some cases, can create a current between the poles. When this electrical field or current is sufficiently strong, and when myocardial cells are disposed within the field or current, the myocardial cells can become depolarized. This depolarization leads to the contraction of the heart muscle. In addition, myocardial cells have the ability to propagate this electrical signal, causing depolarization of adjacent myocardial cells. This self propagation within the myocardium allows a target area of the heart (e.g., the area of the heart corresponding to the right atrium, the right ventricle, the left atrium and/or the left ventricle) to contract upon the stimulation of only a portion of the target area.

Alternatively, or in addition to stimulating the cardiac tissues, in some embodiments the electrodes of the microstimulators of this invention can be configured to sense certain physiological attributes of the heart. For example, the heart's natural electrical signals can be received by an electrode and transmitted to a remote location (e.g., the remote module 12). In addition, other sensing mechanisms that are known in the art can be placed on or near the microstimulators of this application, for example pressure sensors or acoustic sensors.

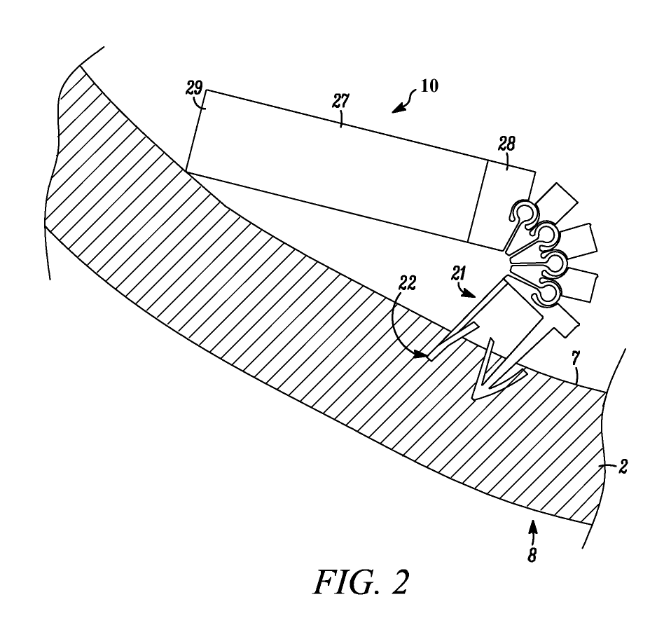

FIG. 2 shows an expanded side view of a microstimulator 10 affixed to cardiac tissue (e.g., myocardium 2) in an implanted configuration according to embodiments of the invention. As shown in FIG. 2, the microstimulator 10 includes a hinged anchor and a control module 27 coupled thereto. The hinged anchor comprises a hinged portion 23 and an anchor portion 21. As further shown and as discussed in greater detail below, the hinged anchor is configured to penetrate or otherwise engage the cardiac tissue (e.g., myocardium 2) to secure the microstimulator 10 thereto, and also to position the control module 27 at a desired orientation when implanted. Additionally, in various embodiments, all or part of the hinged anchor can be configured to operate as an electrode for conducting electrical signals between the microstimulator 10 and the cardiac tissue. The control module 27 can also have portions that can act as an electrode. For example, the control module 27 can have a ring electrode disposed on a portion of the control module 27, or the control module 27 could have one or more barbs or other sharp portions that can penetrate the myocardium 2 and act as an additional electrode surface.

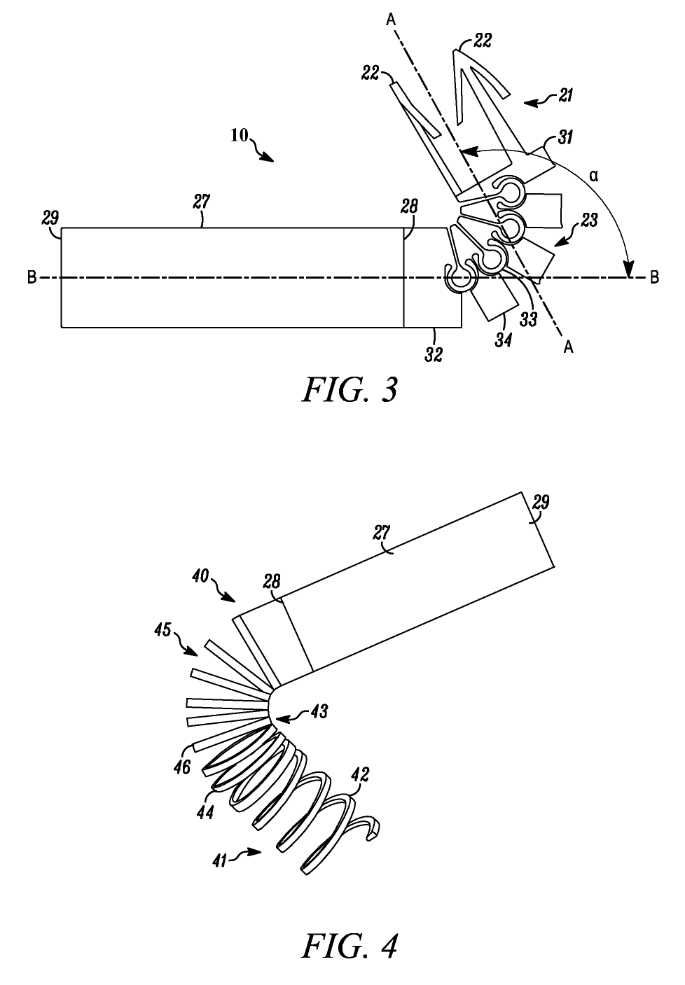

In the illustrated embodiment, the control module 27 includes an outer housing or body having a distal end 28 and a proximal end 29, and defines a longitudinal axis B-B (as further discussed below with respect to FIG. 3). In various embodiments, the control module 27 is configured to generate electrical stimuli to be delivered to the cardiac tissue (e.g., myocardium 2), and also to receive and process the heart's natural electrical signals when operating in a sensing mode. As such, the control module 27 can have a power source disposed therein. Further, the control module or body 27 can also be configured to control the function of the microstimulator 10, and as such the control module 27 can have a controller disposed therein. The control module 27 also includes, in various embodiments, communications components configured to facilitate wireless communication with the remote module 12 (see, e.g., FIG. 1). In some embodiments, all or a portion of the control of the microstimulator 10 can be performed from the control module 27 and/or all or a portion of the control of the microstimulator 10 can be performed from the remote module 12 (see, e.g., FIG. 1) or another remote device.

As mentioned above, the hinged anchor includes an anchor portion 21 and a hinged portion 23. As shown, the anchor portion 21 extends from one end of the hinged portion 23, while the control module 27 is coupled to another end of the hinged portion 23 opposite the anchor portion 21. In the illustrated embodiment, the anchor portion 21 defines a longitudinal axis A-A (as further discussion below with respect to FIG. 3)) and includes a pair of longitudinal members 22 each further including one or more barbs. The longitudinal members 22 are configured to penetrate the cardiac tissue (e.g., myocardium 2) with the barbs fully inserted into the cardiac tissue to impede or substantially prevent spontaneous disengagement of the hinged anchor, and in turn, the microstimulator 10, from its implanted position as illustrated in FIG. 2.

As will be appreciated, because the anchor portion 21 penetrates the cardiac tissue 2, in various embodiments, the anchor portion 21 is configured to operate as an electrode for conducting electrical stimuli and signals between the control module 27 and the cardiac tissue 2. In such embodiments, the anchor portion 21 is electrically coupled, e.g., via the hinged portion 23 or separate leads/conductors as appropriate, to designated circuitry and components in the control module 27. In some examples, other portions of the microstimulator 10 can act as an electrode rather than, or in addition to, the anchor portion 21. For example, a portion of the control module 27 could act as an electrode. Where two electrodes are provided on the microstimulator 10, the microstimulator 10 can be configured as a bipolar system.

As shown in FIG. 2, the hinged portion 23 of the microstimulator 10 can form a bent configuration that allows the control module 27 to be disposed parallel to, or otherwise along, a surface of the heart 1. In some cases, this hinged portion 23 can have a first configuration and a second configuration. In the first configuration, the hinged portion 23 can be in a straight configuration, it can be substantially straight, or it can form a bend. In the second configuration (shown in FIG. 2), the hinged portion 23 can form a bend at a second angle that is sharper than the angle of the hinged portion 23 in the first configuration. These angles are further discussed below with respect to FIG. 3.

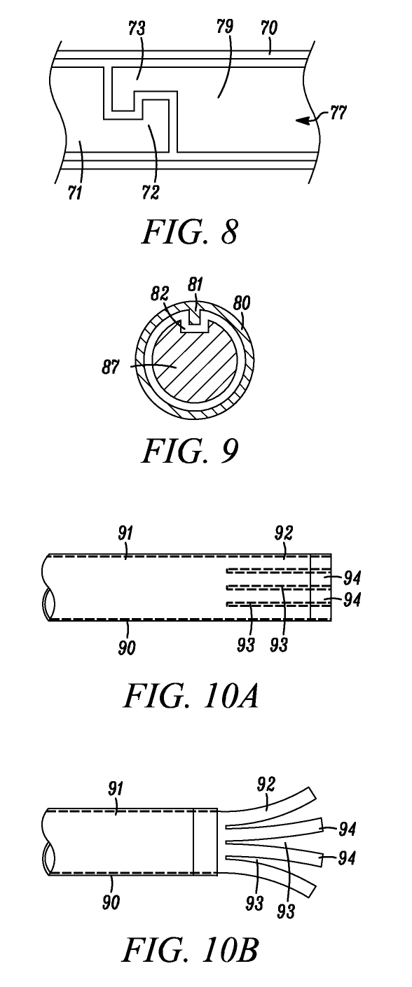

As further discussed below (see, for example, FIGS. 8-13B), the hinged portion 23 can be in a first configuration when microstimulator 10 is being delivered. Upon delivery, the hinged portion 23 of the microstimulator 10 can be predisposed to move from the first to a second configuration. In such cases, the hinged portion 23 can be deflected from the second configuration to form the first configuration. In the first configuration, the microstimulator 10 can be disposed within a delivery system. As such, in the first configuration, the hinged portion 23 can be in a deflected delivery configuration and in the second configuration the hinged portion 23 can be in an undeflected deployed configuration. These configurations are further defined below.

In FIG. 3, the wireless implantable medical microstimulator 10 of FIG. 2 is shown unimplanted and in a second configuration. As shown, a first axis A-A can be defined by the anchor portion 21 of the electrode and a second axis B-B can be defined by the control module 27. In the first configuration, the axes A-A and B-B can be aligned, or substantially aligned, with one another. When the hinged portion 23 is allowed to move from the first to the second configuration, the axes can become less aligned with respect to one another. Further, the hinged portion 23 can be described as nonlinear, bent or angled in the second configuration.

In FIG. 3, the angle that is formed by the hinged portion 23 is shown as the excluded angle α. For the purposes of this application, when the angle α becomes larger, the hinged portion 23 defines a sharper bend or angle. In addition, where the anchor portion 21 and/or the control module 27 are not straight, the axes A-A and B-B can be formed by the initial angle at which the anchor portion 21 and/or the control module 27 take off from their respective ends (31, 32) of the hinged portion.

The angle formed by the hinged portion 23 in the first configuration can be 0-15 degrees and the angle formed by the hinged portion 23 in the second configuration can be 30-180 degrees. One of ordinary skill in the art would be able to determine the particular angles that would be suitable for particular applications.

The hinged portion 23 shown in FIGS. 2 and 3 comprises a series of rings 34 that are separated by U-shaped members 33. The U-shaped members 33 can have an open configuration and a closed configuration. The open configuration can correspond to the first configuration of the hinged portion 23 and the closed configuration can correspond to the second configuration of the hinged portion 23. The U-shaped members 33 can be predisposed to assume the closed configuration.

Each of the U-shaped members 33 can be attached to either two rings 34 or to one ring 34 and one of the ends (31, 32) of the hinged portion 23. Further, the portion of the rings 34 and/or the ends (31, 32) corresponding to the inside of the bend can be shaped to accommodate the bending motion between the first and second configurations. Also, the end 32 that is attached to the control module 27 can be shaped like a flange or other connector in order to facilitate the attachment of the hinged portion 23 to the control module 27.

Further, in some cases such as the embodiment shown in FIGS. 2 and 3, the hinged portion 23 can be predisposed to bend in a predetermined direction. As such, the hinged portion 23 is predisposed to move between first and second configurations in a single plane of movement. (However, when implanted, portions of the anatomy may act upon the microstimulator 10 to move the hinged portion 23 outside of such a predetermined plane of movement.)

As mentioned above, the hinged portion 23 and the anchor portion 21 can together be called a hinged anchor. In some cases, the hinged portion 23 can be separated from the anchor portion 21 and/or the control module 27 by intermediate elements. When intermediate elements are disposed between the hinged portion 23 and the anchor portion 21 and/or the control module 27, the angle formed by the hinged portion 23 is still measured using the angle between the axes A-A and B-B as described above. In this case, if the anchor portion 21 and/or the control module 27 are not straight, the axes A-A and B-B can be defined by the direction that the anchor portion 21 and/or the control module 27 take off from the intermediate element(s).

The anchor portion 21 and the hinged portion 23 can be formed from one unitary structure. For example, a tubular member can be cut or etched to remove portions of the tubular member in order to form the desired anchor and hinged portions. As examples, portions of a tubular member can be removed using EDM, LASER cutting, grinding, chemical etching, or any other suitable process to remove portions of the tubular member. Further, the anchor portion 21 and the hinged portion 23 can be formed separately, for example using any of the above methods, and then joined to one another as shown in the Figures.

In some embodiments, the hinged portion 23 can comprise an elastic (i.e., linear elastic) or superelastic material, for example an alloy such as Nitinol (which can be either superelastic or linear elastic), Elgiloy®, or other suitable alloys The hinged portion 23 can also comprise an alloy that has shape memory properties at or near human body temperature (e.g., shape memory Nitinol). The hinged portion 23 could also comprise any other suitable elastic material, such as an elastic polymer. In some cases, the hinged portion 23 can comprise an elastic or superelastic material as mentioned above and the anchor portion 21 can comprise a relatively inflexible, inelastic or malleable material, for example a stainless steel, a cobalt-chromium alloy such as MP35N, Titanium, or any other suitable material.

Further, in any of the embodiments described herein, the hinged portion and/or the anchor portion can have a therapeutic coating material disposed over at least a part of the hinge and/or anchor portions. Such coatings can have antithrombogenic, anti-inflammatory, immunosuppressant, or other properties known in the art. In some cases, the coating can comprise a drug-eluting material that can elute a therapeutic agent, for example heparin, a steroid, or immunosuppressant agents such as dexamethazone.

The hinge portion and/or the anchor portion can also have coatings with particular physical properties. For example, the coatings could have insulative or lubricious or other properties known in the art. In embodiments where electrical energy is transmitted through the hinged portion to the anchor portion, an outer portion of the hinged portion may be electrically insulated in order to ensure that the electrical energy is transmitted to the anchor portion rather than to the ambient surroundings.

In some embodiments, the hinged portion 23 and the anchor portion 21 can comprise the same material and the material can be treated in order to provide different properties in different portions of the device. For example, the hinged anchor can comprise a Nitinol that has been treated to be linear elastic or superelastic in the hinged portion 23 and relatively inflexible, inelastic or malleable in the anchor portion 21. In some embodiments, the hinged portion 23 can be manufactured (i.e., formed from a tubular member as mentioned above) and then the hinged portion 23 can be set (e.g., heat set) into a second, bent configuration so that the hinged portion 23 can be predisposed to assume a second, bent configuration. In other embodiments, the hinged portion and/or the anchor portion can be made by forming the desired hinged and/or anchor portion shape in a flat sheet of material and subsequently rolling the flat sheet to form the hinged and/or anchor portion. Further, the hinged and/or anchor portion can be made from flat or round wire that can be shaped into any of the hinged and/or anchor portions discussed herein.

FIG. 4 shows a perspective view of a leadless microstimulator electrode according to embodiments of the invention. The microstimulator 40 can comprise a control module 27, a hinged portion 43 and an anchor portion 41. As above, the hinged portion 43 and the anchor portion 41 can together define a hinged anchor.

The hinged portion 43 can have a distal end 44 and a proximal end 45, and can comprise a helical coil 46. The first and second configurations of the hinged portion 43 and the manner in which the hinged portion 43 can deflect between the first and second configurations can be the same as disclosed above with respect to hinged portion 23. Further, the hinged portion distal end 44 can have an anchor portion 41 disposed thereon. For example, the anchor portion 41 can comprise a coil 42. The coil 42 can be a continuation of the coil of the hinged portion 43, or the coil 42 can be a separate coil that can be attached to the hinged portion distal end 44. The coils of the hinged portion 43 and the anchor portion 41 can be made of the same material, or they can comprise different materials. The materials of construction and the manner of producing the hinged anchor of FIG. 4 can be similar to the materials and methods discussed above with respect to FIGS. 2 and 3.

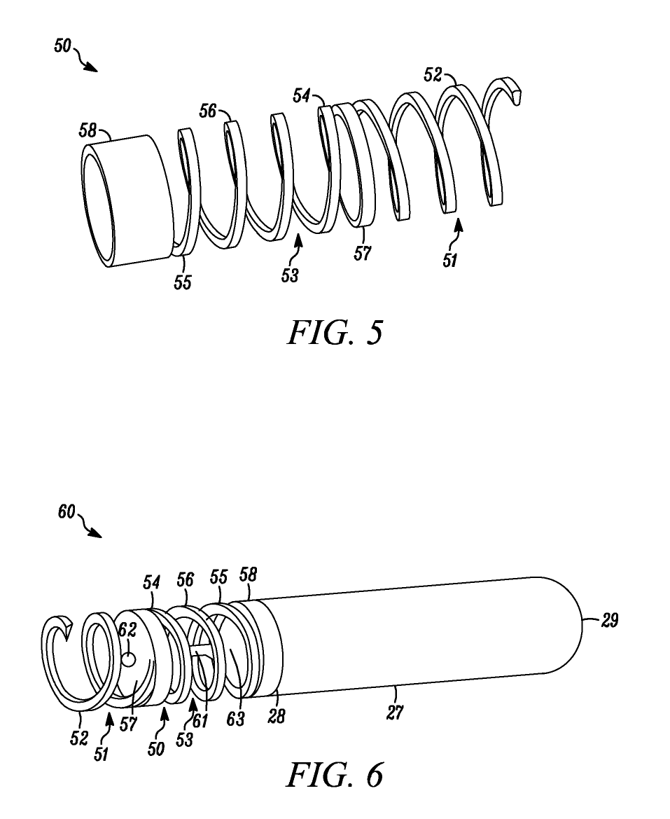

FIG. 5 shows a perspective view of a hinged anchor 50 according to additional embodiments of the invention. The hinged anchor 50 can have an anchor portion 51 and a hinged portion 53. The anchor portion 51 can comprise a coil 52. The hinged portion 53 can comprise a coil 56 and can have a distal end 54 and a proximal end 55. Similar to the embodiments of FIGS. 2-4, the hinged portion 53 can have a proximal flange or collar 58 disposed at the hinged portion proximal end 55. This flange or collar 58 can facilitate the attachment of the hinged portion 53 to the control module 27. In addition, the hinged portion 53 and the anchor portion 51 can be connected via a connector 57, for example a connector ring or disc 57.

The materials of construction and the manner of producing the hinged anchor of FIG. 5 can be similar to the materials and methods discussed above with respect to FIGS. 2-4. As mentioned above with respect to FIGS. 2-4, the coil 56 of the hinged portion 53 can be predisposed to assume a nonlinear configuration. However, in some cases, as discussed below with respect to FIG. 6, the coil 56 of the hinged portion 53 can be predisposed to assume a straight or substantially straight configuration.

FIG. 6 shows the hinged anchor 50 of FIG. 5 disposed on a control module 27. In this embodiment, the coil 56 of the hinged portion 53 can be predisposed to assume a straight configuration. Further, the hinged portion 53 can comprise the coil 56 and a compression member 61. The compression member 61, shown in this example as a wire or cable, can extend from the control module 27 to the connector 57. A compression member distal end 62 can connect to the connector 57. For example, the compression member distal end 62 can extend through an opening in a connector disc 57. The compression member distal end 62 can be made larger than, or can have a different shape than, an opening in the connector disc 57 so that the compression member 61 cannot be pulled back through the opening in the connector disc 57. In this way, the compression member 61 can maintain the coil 56 of the hinged portion 53 in a compressed state.

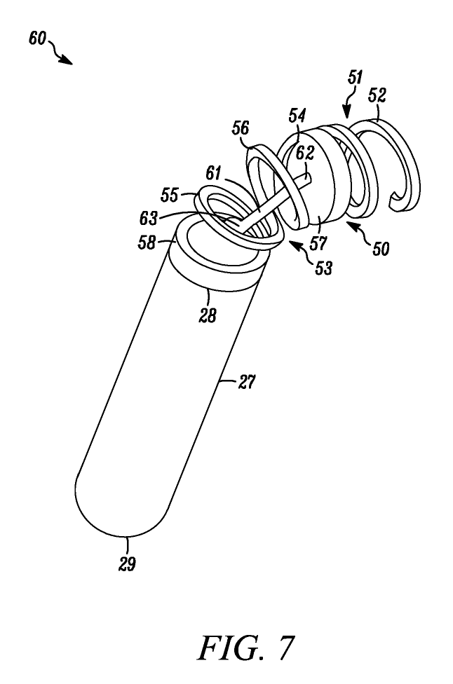

As shown in FIG. 7, because the coil 56 of FIG. 6 is in a compressed state, the hinged portion 53 can be predisposed to form a second configuration that is bent at an angle. The angle formed in the second configuration can be sharper than an angle that is formed in a first configuration, for example the first configuration shown in FIG. 6. The angles formed by the hinged portion 53 in the first and second configurations can be any of the angles discussed above with respect to FIGS. 2 and 3.

In the case of the hinged anchor 50, the hinged portion 53 is not predisposed to bend in any particular direction when moving between the first and second configurations. The coil 56 that is under compression can form a second configuration in any direction, for example depending on the anatomy in which the microstimulator 60 is implanted. In some cases, if the compression member 61 extends through the connector 57 at a location that substantially balances the forces of the coil 56, the hinged portion 53 will have substantially no preference in the direction of bending. Such a design can allow the microstimulator 60 to assume a second configuration that is determined by the local anatomy in which it is implanted, and can further reduce the interference between the microstimulator 60 and a patient's anatomy.

In some embodiments, the amount of compression that the coil 56 is placed under can affect how sharp the hinged portion 53 will bend in a second configuration. For example, greater compression of the coil 56 can facilitate the hinged portion 53 forming a sharper bend in the second configuration.

FIGS. 8-10B show delivery systems according to embodiments of the invention. In FIG. 8, a delivery system is shown comprising a tubular delivery member 70 with an actuator 71 disposed therein. A control module 77 is shown disposed in the tubular delivery member 70. The control module 77 can be similar in most respects to the other control modules described herein. The actuator 71 has a distal end 72 with a first keyed portion and the control module 77 has a second keyed portion 73 adjacent the control module proximal end 79. The first and second keyed portions can be shaped and configured to mechanically fit together. As shown in FIG. 8, the keyed portions can mechanically interact to transmit axial forces (pushing forces and/or pulling forces) and/or torque from the actuator 71 to the control module 77. In cases where the anchor portion of the microstimulator can be screwed into body tissue (e.g., those embodiments shown in FIGS. 4-7), the actuator 71 can be rotated in order to turn the control module 77, which can in turn rotate the anchor portion, facilitating implantation of the microstimulator. Also, in some cases where the hinged portion deflects in a predetermined direction (e.g., in the embodiments shown in FIGS. 2-4), it may be desirable to rotate the microstimulator in order to ensure that the direction of bending for the microstimulator minimizes interference with the local anatomy.

FIG. 9 shows a cross-sectional view of another delivery system according to embodiments of the present invention. A tubular delivery member 80 can have a control module 87 disposed therein. The control module 87 can be similar in most respects to the other control modules described herein. The tubular delivery member 80 can have a first keyed member 81 disposed on an inside surface of the tubular delivery member 80. The control module 87 can have a second keyed member 82 disposed on an outside surface of the control module 87. The keyed member 81 can extend the entire length of the tubular delivery member 80, or it can extend along only a portion of the tubular delivery member 80 (e.g., a distal portion of the tubular delivery member 80). In addition, keyed member 82 can extend along all, or only a portion of, control module 87. The keyed members can mechanically interact to transmit torque from the tubular delivery member 80 to the control module 87. Again, this can facilitate positioning of the control module 87 in a desired orientation and/or facilitate the implantation of the microstimulator in a target area of body tissue. The delivery device of FIG. 9 can also have an actuator member to push the microstimulator out of the tubular delivery member 80. In some cases, the actuator member can be similar to the actuator member shown in FIG. 8, and the control module 87 can be keyed to both the actuator distal end and the tubular delivery member 80.

In some cases, the keyed structures of FIG. 9 can be reversed. In other words, a keyed portion on the control module 87 can extend into a keyed depression on the tubular delivery member 80. Further, in some embodiments, the shapes and sizes of the control module 87 and the lumen of the tubular delivery member 80 can be such that the control module 87 cannot turn freely within the tubular delivery member 80. In such cases, the entire control module 87 is essentially keyed in the tubular delivery member 80 and turning the tubular delivery member 80 can cause the control module 87 to rotate.

FIGS. 10A and 10B show a delivery device in accordance with additional embodiments of the invention. The delivery system has an outer tubular member 90 and an inner tubular member 91. The inner tubular member 91 can have a distal end 92 with longitudinal cuts 93 formed therein. The cuts 93 can form strips 94 of material, and the strips 94 can be predisposed to assume an open position (as shown in FIG. 10B). When the strips 94 are disposed in the outer tubular member 90, they can be captured in a closed configuration shown in FIG. 10A and when the strips 94 are extended outside of the outer tubular member 90, they can assume a second open position shown in FIG. 10B. The inner tubular member 91 can be sized such that, when the strips 94 are in a closed position, a portion of a microstimulator can be captured within the inner tubular member 91. In this way, microstimulators can be captured within the inner tubular member 91 and delivered to a target area.

The strips 94 can, in some cases, grip the microstimulator tightly enough to allow the microstimulator to be rotated by rotating the inner tubular member 91, facilitating positioning of the microstimulator and/or implantation of the microstimulator. Further, the delivery device of FIGS. 10A and 10B can have an actuator member disposed within the inner tubular member 91 which can be used for pushing the microstimulator out of the inner tubular member 91. The delivery device of FIGS. 10A and 10B can also have a keyed microstimulator and a keyed actuator member and/or a keyed inner tubular member 91, as described above with respect to FIGS. 8 and 9. Incorporating the structures shown in FIGS. 8 and 9 can help ensure sufficient torque transmission to the microstimulator in order to facilitate placement and/or implantation of the microstimulator.

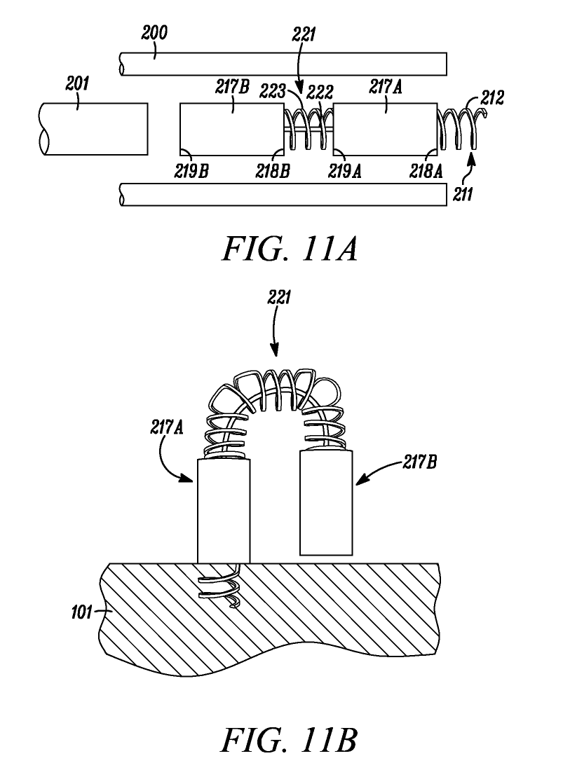

FIGS. 11A and 11B show additional embodiments of the present invention. In FIG. 11A, a microstimulator is shown disposed in a delivery system. The delivery system comprises a tubular delivery member 200 and an actuator 201. The delivery system shown in FIG. 11A can have features similar to any of the delivery systems described with respect to FIGS. 8-10B above. For example, a proximal portion 219b of a control module 217b can be keyed to mechanically engage the actuator 201 or the control modules 217a, 217b can be keyed to mechanically engage the tubular delivery member 200, as mentioned above with respect to FIGS. 8-10B.

The microstimulator can comprise an anchor portion 211, a hinged portion 221 and control modules 217a, 217b. The anchor portion 211 is shown as a coil anchor similar to the anchor portions shown in FIGS. 4-7, although any other anchor structure can be used (e.g., the anchor portion shown in FIGS. 2 and 3). The control modules 217a, 217b can be the same as the control modules discussed above, although the functionality of the control modules discussed above can be split between the two control modules 217a, 217b. For example, a power supply can be disposed in control module 217a and a controller can be disposed in control module 217b, or vice versa. In addition, each of the control modules 217a, 217b can have both a power supply and a controller. In some instances, a surface of each of the control modules 217a, 217b can function as an electrode surface, and in this manner the microstimulator shown in FIGS. 11A and 11B can be a bipolar system.

Further, the hinged portion 221 shown in FIGS. 11A and 11B is a compressed coil hinged system, similar to the hinged portion described with respect to FIGS. 5-7. This hinged portion 221 can have a coil 223 that is kept under compression by compression member 222. The hinged portion 221 can connect a distal end 218b of one of the control modules 217b to a proximal end 219a of the other control module 217a. Other hinged portions can also be used between the control modules 217a, 217b, for example the hinged portions shown in FIGS. 2-4.

In some embodiments, the microstimulator can have more than one hinged portion. For example, a first hinged portion can be disposed between the two control modules (as shown in FIGS. 11A and 11B) and a second hinged portion can be disposed between the control module 217a and the anchor portion. The first hinged portion can be predisposed to bend in a first plane and the second hinged portion can be predisposed to bend in a second plane. The planes can be different planes, and in some cases the planes can be perpendicular or substantially perpendicular to one another.

FIG. 11B shows the microstimulator after it has been implanted in the myocardium 2 and released from the delivery system. Upon release from the delivery system, the microstimulator can deflect between a first configuration (shown in 10A) and a second configuration (shown in 10B). The microstimulator can be predisposed to assume the second configuration. The first and second configurations can be defined by the angle between the anchor portion 211 and the control module 217b, as discussed above with respect to FIGS. 2 and 3.

Depending on the type of hinged portion 221 that is employed in the microstimulator, a deployed microstimulator can be predisposed to assume a second configuration with an angle of about 180 degrees as shown in FIG. 11B. In some cases, the angles of the first and second configurations can be any of the angles for the first and second configurations disclosed with respect to FIGS. 2 and 3. Also, in some embodiments, the control modules 217a, 217b can be magnetically attracted to one another. This magnetic attraction can further facilitate the predisposition of the microstimulator to assume a second configuration such as the second configuration shown in FIG. 11B.

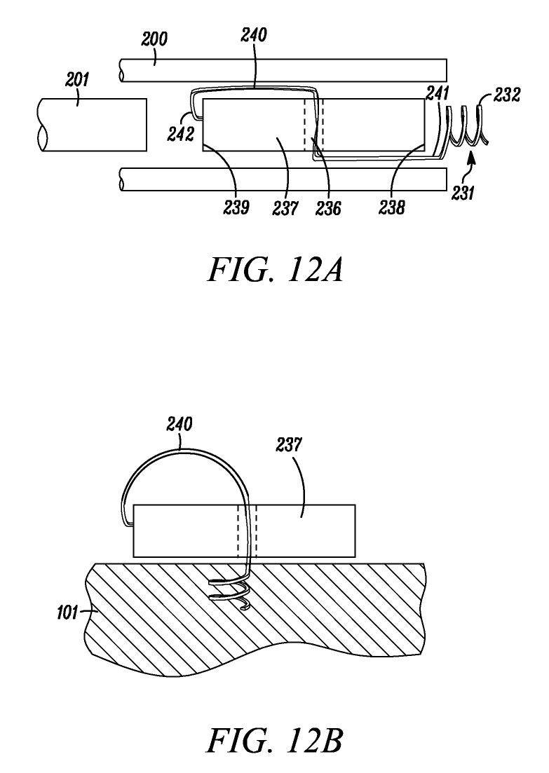

FIGS. 12A and 12B show another microstimulator design according to embodiments of the invention. The microstimulator can have an anchor portion 231, a control module 237 and a hinged portion 240. The control module 237 can have a distal end 238, a proximal end 239 and a channel 236. The channel 236 can be formed through the control module 237 near the center of the control module 237. The control module 237 can otherwise be similar to any of the control modules described herein.

In FIG. 12A, the microstimulator is shown disposed in a delivery system. The delivery system can be similar to the delivery system described with respect to FIG. 11A; for example, the delivery system of FIG. 12A can be any of the delivery systems described above with respect to FIGS. 8-10B. In some cases, the control module 237 can mechanically interact with keyed portions of the tubular delivery member 200 and/or the actuator 201, also as described with respect to FIGS. 8-10B.

The anchor portion 231 is shown as a coil 232, but, again, this anchor portion 231 can be any suitable anchor design, for example the barbed anchor shown in FIGS. 2 and 3. The anchor member 232 can be attached to a distal end 241 of the hinged portion 240. The hinged portion proximal end 242 can be attached to the control module 237. In a first configuration (as shown in FIG. 12A), the anchor portion 231 and the control module 237 can be generally aligned, or form an angle of about zero degrees (the angle can be measured as described above with respect to FIG. 3). In other embodiments, the angle formed in this first configuration can be any of the first configuration angles disclosed above with respect to FIGS. 2 and 3.

In the first configuration, the hinged portion 240 can extend from the anchor portion 231 along the control module 237 and through the channel 236. A hinged portion proximal end 242 can be attached to the control module 237 at an attachment point (e.g., an attachment point at the control module proximal end 239 shown in FIG. 12A).

As shown in FIG. 12B, the microstimulator of FIG. 12A can also assume a second configuration and can be implanted in body tissue (i.e., implanted in the myocardium 2). In this second configuration, the anchor portion 231 and the control module 237 can be at a second angle with respect to one another. For example, this second angle can be any of the second configuration angles disclosed above with respect to FIGS. 2 and 3.

Further, the hinged portion 240 can be predisposed to assume the second configuration. For example, any of the processing methods discussed herein (heat-setting, etc) can be used to predispose the hinged portion in a particular second configuration. As shown in FIG. 12B, the hinged portion 240 can be predisposed to pull itself through the channel 236, forcing the control module 237 into a position near the anchor portion 231. In some cases, the hinged portion 240 in the second configuration can form a looped portion as shown in FIG. 12B.

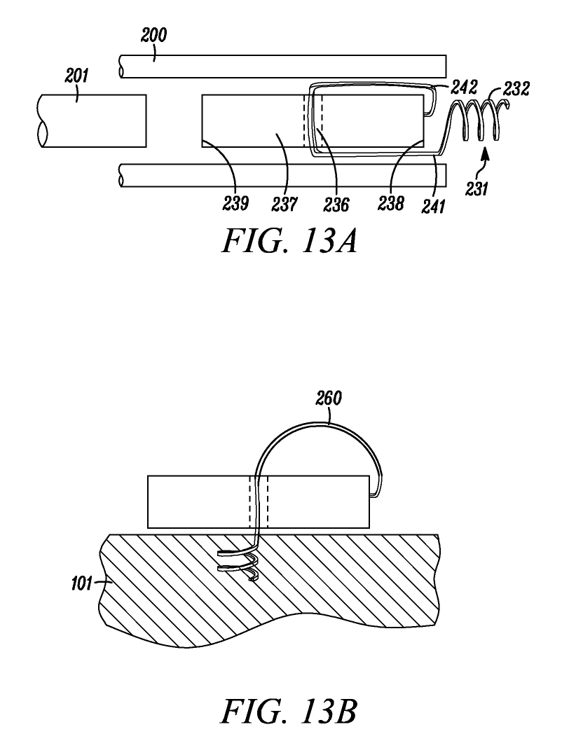

FIGS. 13A and 13B show another microstimulator according to embodiments of the invention. The microstimulator shown in these figures can be substantially similar to the microstimulator shown in FIGS. 12A and 12B except that the proximal end of the hinged portion 260 can attach to the control module distal end 238.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A leadless microstimulator comprising:
an anchor configured to penetrate and engage tissue at an implantation site, the anchor defining a first longitudinal axis;
a control module configured to generate an electrical stimulus, the control module defining a second longitudinal axis;
a hinge disposed between the anchor and the control module, the hinge having a first configuration and a second configuration, wherein in the first configuration the first and second longitudinal axes are more closely aligned than in the second configuration, wherein the hinge is predisposed to assume the second configuration and the hinge is deflectable between the first and second configurations.

2. The leadless microstimulator of claim 1, wherein a first side of the hinge is attached to the anchor and a second side of the hinge is attached to the control module.

3. The leadless microstimulator of claim 1, wherein the anchor portion comprises a coil.

4. The leadless microstimulator of claim 1, wherein the anchor portion comprises barbs.

5. The leadless microstimulator of claim 1, wherein the hinged portion comprises a coil.

6. The leadless microstimulator of claim 1, wherein the hinged portion comprises at least one U-shaped member that has an open configuration and a closed configuration, the open configuration corresponding to the first configuration and the closed configuration corresponding to the second configuration, the U-shaped member predisposed to assume the closed configuration.

7. The leadless microstimulator of claim 1, wherein the hinged portion is predisposed to bend in a predetermined direction when bending between the first and second configurations.

8. The leadless microstimulator of claim 1, wherein the hinged portion is not predisposed to bend in any one direction when bending between the first and second configurations.

9. The leadless microstimulator of claim 1, wherein the hinged portion is a coil that is held in compression.

10. The leadless microstimulator of claim 1, wherein the anchor portion comprises an electrode.

11. The leadless microstimulator of claim 1, the microstimulator further comprising a second control module, wherein the hinged portion is disposed between the first and second control modules.

12. An electrode for a leadless microstimulator, the electrode comprising:
a hinged anchor having an anchor portion and a hinged portion;
wherein the hinged portion has a first configuration and a second configuration;
wherein the hinged portion forms a first angle in the first configuration and a second angle in the second configuration, the second angle being sharper than the first angle; and
wherein the hinged portion is predisposed to assume the second configuration.

13. The electrode of claim 12, wherein the anchor portion comprises a coil.

14. The electrode of claim 12, wherein the anchor portion comprises barbs.

15. The electrode of claim 12, wherein the hinged portion comprises a coil.

16. The electrode of claim 12, wherein the hinged portion comprises at least one U-shaped member that has an open configuration and a closed configuration, the open configuration corresponding to the first configuration and the closed configuration corresponding to the second configuration, the U-shaped member predisposed to assume the closed configuration.

17. The electrode of claim 12, wherein the hinged portion is predisposed to bend in a predetermined direction when bending between the first and second configurations.

18. The electrode of claim 12, wherein the hinged portion is not predisposed to bend in any one direction when bending between the first and second configurations.

19. The electrode of claim 12, wherein the hinged portion is a coil that is held in compression.

20. A microstimulator comprising a control module, an anchor portion and a flexible hinge, wherein the flexible hinge is disposed between the control module and the anchor portion, and wherein the hinge is predisposed to form a nonlinear configuration.

21. The microstimulator of claim 20, wherein the flexible hinge is deflectable to a substantially straight configuration for delivery.

22. The microstimulator of claim 20, wherein a first side of the hinge is attached to the control module and a second side of the hinge is attached to the anchor portion.

23. The microstimulator of claim 20, further comprising a second control module, wherein the hinge is disposed between the first and second control modules.
